(19) United States
(12) Patent Application Publication
(10) Pub. No.: US 2017/0304076 A1
(43) Pub. Date: Oct. 26, 2017

(54) SPACER FABRICS FOR USE IN A SPINAL IMPLANT DEVICE

(71) Applicant: Arthrex, Inc., Naples, FL (US)

(72) Inventors: Matthew Palmer, Medford, MA (US); Matthew Fonte, Concord, MA (US); Robert Devaney, Auburndale, MA (US)

(21) Appl. No.: 15/486,510

(22) Filed: Apr. 13, 2017

Related U.S. Application Data
(60) Provisional application No. 62/325,312, filed on Apr. 20, 2016.

(51) Int. Cl. A61F 2/44; A61F 2/30

(52) U.S. Cl. CPC A61F 2/4465; A61F 2002/30092; A61F 2002/4495

(57) ABSTRACT

A device promotes osseointegration between two adjacent vertebrae. The device includes a spacer fabric having a top layer, a bottom layer, and intermediate filler fibers connecting the top layer and the bottom layer. The spacer fabric is capable of expanding to fill a gap between two adjacent vertebrae.

### CROSS REFERENCE TO RELATED APPLICATION

The application claims priority to U.S. Provisional Application No. 62/325,312 which was filed on Apr. 20, 2016, the entire disclosure of which is hereby incorporated by reference.

### BACKGROUND OF THE INVENTION

In the field of spinal surgery, it is common to fuse adjacent vertebrae. The success of a surgical procedure often depends on the ability to rigidly fix the adjacent vertebrae and to pack bone or other biologically active materials between the vertebrae to act as a bridge. Often, a bone graft (e.g., iliac crest bone) or calcium phosphate/hydroxyapetite is used. The bone graft not only aids in osteointegration, but also helps to replace the space that was occupied by the intervertebral disk. Spinal cages may be used to occupy the gap, provide sufficient mechanical strength to keep the adjacent vertebrae spaced as desired, and can be packed with biologically active materials to aid in fusion.

While biologically active agents are useful for bone fusions, it can be difficult to localize and maintain them in place. While cages are good at holding biologically active agents in place, they do not fill the entire footprint of the removed intervertebral disk. Therefore, gaps exist between the two vertebrae, inhibiting osteointegration. A need exists for a device that is able to localize biologically active materials, while providing full contact between the two adjacent vertebrae and maintaining vertebral spacing.

Additionally, in the field of spinal surgery, it is often necessary to remove diseased or damaged intervertebral disks. Disk degeneration is a common disease. The removed disk may be replaced with a synthetic disk or an allograft disk. Alternatively, the disk may be removed, and the adjacent vertebrae fused. Intervertebral disks act as shock absorbers, dampening the loads applied to the spinal column.

Unfortunately, allograft disks and synthetic disks can fail. The disks can fatigue and lose their resilience. There is a clinical need for synthetic disks that resist fatigue.

### SUMMARY

A spinal implant that utilizes spacer fabrics is disclosed herein.

A spacer fabric is manufactured from a shape memory material (e.g., a material capable of exhibiting superelasticity and/or a temperature-induced shape change). The shape memory material may be a metal alloy (e.g., Nitinol) or a polymer (e.g., appropriately processed PEEK, or polyether ether ketone).

Spacer fabrics are a generic term for three dimensional fabrics that have a top layer, a bottom layer, and a middle layer that interconnects the top layer and the bottom layer. Spacer fabrics are commonly used in many industries and are often used in applications where fluid flow, cushioning, and vibration absorption are necessary. Spacer fabrics may be manufactured using knitting or weaving techniques. Currently, spacer fabrics are manufactured of monofilament polymeric yarns, polyamide, or polyester fibers. These materials are highly flexible, kink resistant, and common in the textile field.

The spacer fabrics can use a shape memory material (SMM). Shape memory materials, unlike other metallic filaments, are highly flexible and kink resistant, allowing them to be woven or knit. This class of spacer fabrics is substantially stronger than polymer monofilaments and is not susceptible to deleterious creep and fatigue, which can shorten the life of polymeric materials. Shape memory material spacer fabrics can be designed to be strong and superelastic. They exhibit a hysteresis for large shape recovery strains and can be designed to change shape based on temperature changes. Furthermore, unlike polymers, metallic shape memory materials have been shown to promote bone ongrowth.

In one example, the spacer fabric is designed as an intervertebral fusion device. The spacer fabric promotes osteointegration between adjacent vertebrae. The spacer fabric can be engineered so that a pore size is optimized for osteointegration (e.g., pore sizes between 150 μm and 750 μm). Furthermore, the pores may be filled with a bone graft at the time of surgery. Alternatively, the spacer fabric can be pre-impregnated with a biologically active agent (e.g., calcium phosphate). It is possible to fill the spacer fabric with a slurry of calcium phosphate and methyl cellulose and then lyophilize the resulting structure to form a hybrid structure elastic structure.

The three-dimensional nature of the spacer fabric is ideal for fusion procedures. The spacer fabric can be made at a desired thickness (e.g., the thickness of the intervertebral disk that was removed). Unlike a cage, the shape memory material spacer fabric will conform to the anatomy of the vertebrae, expanding to fill any gaps.

The three-dimensional spacer fabric can be implanted as a sheet and cut to the footprint of the intervertebral space. Alternatively, the spacer fabric can be rolled into a log and used like a traditional cage. Furthermore, the shape memory material spacer fabric can be impregnated with a biologically active agent and put into a traditional cage to further aid in localizing the biologics effect.

Furthermore, a traditional intervertebral fusion device may have a coating of shape memory material spacer fabric on its surface. This will allow for osseointegration. Many fusion devices are made from PEEK, which has been shown to not encourage bone in/on-growth. A PEEK fusion device with a shape memory material spacer fabric coating can provide the benefit of a PEEK cage with the osseointegration properties of the spacer fabric.

In another application, the spacer fabric is designed to act as an intervertebral disk and provides cushioning between adjacent vertebrae. The spacer fabric may be homogenous in mechanical properties or it may have regions of differing mechanical properties. When made of a shape memory material, the spacer fabric is highly crush resistant.

The spacer fabric intervertebral disk can be placed directly adjacent to the vertebrae or it can be fixed to plates that engage the adjacent vertebrae.

The knit material, the density, and the filament size may vary across the intervertebral disk. It may be beneficial to have a very tough outer annulus and a less dense nucleus. The annulus may be made of a bio-resorbable material. The removed annulus may be ground up and impregnated into the disk to promote regrowth.

The spacer fabric intervertebral disk may be partially resorbable and partially non-resorbable.

### BRIEF DESCRIPTION OF THE DRAWINGS

FIG. 1 is a schematic view detailing the general structure of a spacer fabric;

FIG. 2 is a schematic view of an intervertebral fusion device that can be manufactured from the spacer fabric;

FIG. 3 is a schematic view of a spinal cage that can be made of the spacer fabric; and

FIG. 4 is a schematic view of an intervertebral disk that can be made of the spacer fabric.

### DETAILED DESCRIPTION

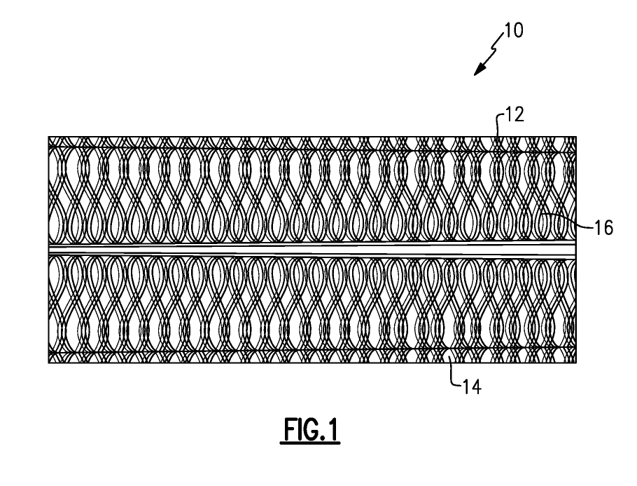

FIG. 1 is a schematic view detailing the general structure of a spacer fabric 10. The spacer fabric 10 includes a top layer 12, a bottom layer 14, and filler fibers 16 interconnecting the top layer 12 and the bottom layer 14. A spacer fabric 10 is often used in applications where fluid flows, cushioning, and vibration absorption are necessary.

When used as a porous coating for an implant, the three layers 12,14 and 16 of the spacer fabric 10 have three different functions. For example, the top layer 12 provides for bone ingrowth, the filler fibers 16 provide for elasticity, and the bottom layer 14 provides for brazing to the implant.

The top layer 12 of the spacer fabric 10 can have a structure of repeating dodecahedrons, i.e., a honeycomb geometry similar to that of cancellous bone, to facilitate osseointegration. The bottom layer 14 of the spacer fabric 10 can be engineered to facilitate braising to the implant. The filler fibers 16 of the spacer fabric 10 provide spring and is knit vertical (e.g., at an angle of 30°-150°) from the top layer 12 and the bottom layer 14.

The spacer fabric 10 is three-dimensional in construction, and filler fibers 16 create the elastic response for the spacer fabric 10 when compressed, and/or made to bend, and then allowed to recover. The three-ply structure of the spacer fabric 10 has good breathability, wettability, crush resistance, and a three-dimensional porous appearance, which makes it ideal for use as a dynamic porous coating. Each layer 12, 14 and 16 of the spacer fabric 10 can be made of different materials and have different porosity levels and geometry. Layers spacer fabric 10 can be stacked one on top of another to form a multi-level spacer fabric construct.

The three distinct layers 12, 14 and 16 of the spacer fabric 10 can be manufactured using three distinct wire sizes. As an example, it is possible to use a large wire size for the bottom layer 16 so as to increase the surface area available for bonding to the implant, a medium wire size for the filler fibers 16 to give the spacer fabric 10 appropriate stiffness and elasticity, and a fine wire size for the top layer 12 (i.e., the surface contacting the bone) to better match the cancellous bone structure.

The spacer fabric 10 includes a shape memory material (SMM). Shape memory materials, unlike other metallic filaments, are highly flexible and kink resistant. This allows the shape memory material to be woven or knit. The spacer fabric 10 is substantially stronger than polymer monofilaments and is not susceptible to deleterious creep and fatigue, which often shortens the life of polymeric materials. Shape memory material spacer fabrics can be designed to be strong and superelastic. These fabrics exhibit a hysteresis for large shape recovery strains and can be designed to change shape based on temperature changes. Additionally, unlike polymers, metallic shape memory materials have been shown to promote bone ingrowth.

The spacer fabric 10 may be manufactured using knitting or weaving techniques. In one example, the spacer fabric 10 is manufactured using monofilament polymeric yarns, polyamide, or polyester fibers. These materials are highly flexible and kink resistant.

The spacer fabric 10 is manufactured from a shape memory material (e.g., a material capable of exhibiting superelasticity and/or a temperature-induced shape change). The shape memory material may be a metal alloy (e.g., Nitinol) or a polymer (e.g., appropriately processed polyether ether ketone (PEEK)).

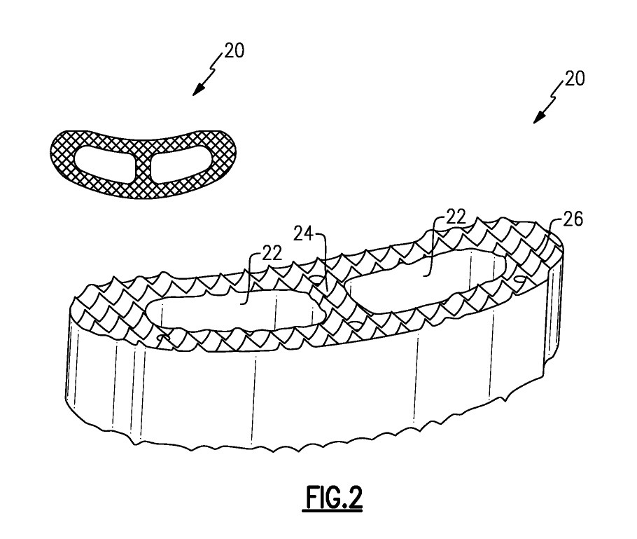

FIG. 2 is a schematic view of an intervertebral fusion device 20 (spinal implant) that can be manufactured from a spacer fabric 10 of a shape memory material. The spacer fabric 10 promotes osteointegration between adjacent vertebrae. The spacer fabric 10 can be engineered so that a pore size is optimized for osteointegration (e.g., pore sizes between 150 μm and 75 μm). Furthermore, pores may be filled with bone graft at the time of surgery. Alternatively, the spacer fabric 10 can be pre-impregnated with a biologically active agent (e.g., calcium phosphate). The spacer fabric 10 can be filled with a slurry of calcium phosphate and methyl cellulose and then lyophilized such that the resulting structure forms a hybrid elastic structure.

The intervertebral fusion device 20 can be made in the illustrated shape or other shapes to fill a footprint between two vertebrae. As illustrated, intervertebral fusion device 20 has a curved shape. The intervertebral fusion device 20 includes two openings 22 that are separated by a piece of material 24. Additional material 26 surrounds the two openings 22. In one example, the intervertebral fusion device 20 can be made of polyether ether ketone (PEEK) and coated with the spacer fabric 10. The spacer fabric 10 is capable of expanding or laterally moving to fill gaps between the adjacent vertebrae and create an interference fit. In one example, the spacer fabric 10 can be impregnated with a biologically active agent. In another example, the biologically active agent includes hydroxyapatite or calcium phosphate.

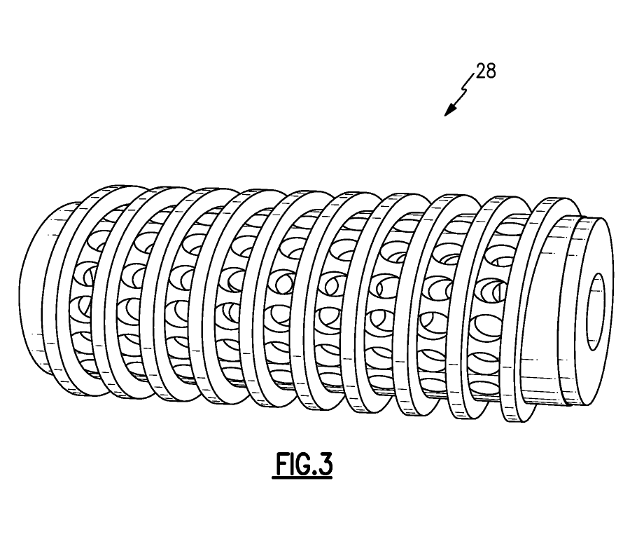

FIG. 3 is a schematic view of a spinal cage 28 that can be made of the spacer fabric 10. The spinal cage 28 can be made by rolling the spacer fabric 10 into a roll and using the roll as a traditional cage. Alternatively, the rolled spacer fabric 10 can be put inside a standard cage. Furthermore, the shape memory material spacer fabric 10 can be impregnated with a biologically active agent and put into a traditional cage to further aid in localizing the biologics effect. The three-dimensional spacer fabric 10 can also be implanted as a sheet and be cut to the footprint of the intervertebral space.

The three-dimensional nature of the spacer fabric 10 of the spinal cage 28 can be utilized for a fusion procedure. The spacer fabric 10 can be made of a desired thickness (e.g., the thickness of the intervertebral disk that was removed). Unlike a cage, the shape memory material spacer fabric 10 will conform to the anatomy of the vertebrae, expanding to fill any gaps.

In another example, a traditional intervertebral fusion device or cage can have a coating of shape memory material spacer fabric 10 on its surfaces. The coating will allow for osseointegration of the cage. Many fusion devices are made from PEEK, which has been shown to hinder bone in/on-growth. A PEEK fusion device or cage with a shape memory material spacer fabric 10 coating can provide the benefit of the PEEK cage while having the osseointegration properties of the spacer fabric 10.

The coating of the spacer fabric 10 can be made of knitted shape memory wire. Knit structures, with their high number of individual fibers, allow for the creation of very intricate porous patterns, increasing the performance capabilities for the dynamic porous coating.

Warp and weft are two different types of knitting which may be used to form a dynamic porous coating. Warp knits have fibers that extend along the length of the material. Weft knits have fibers that extend across the width of the material.

Knits can be created in either sheets or tubes. Multiple sheets can be laminated on top of one another, and tubes can be formed concentric to one another in order to achieve a more three-dimensional structure for the dynamic porous coating. In addition, by laminating sheets or tubes with different porosities, a more complex overall pore structure can be created for the dynamic porous coating.

Another method for manufacturing a dynamic porous coating is to use an additive manufacturing technique. The dynamic porous coating may be brazed to an implant. Brazing is a metal-joining process whereby a filler metal is heated above its melting point and distributed between two or more close-fitting parts by capillary action. The filler metal is brought to slightly above its melting (liquidus) temperature while protected by a suitable atmosphere, usually a flux, a vacuum, or an inert atmosphere. The filler metal then flows over the base metal, known as wetting, and is then cooled to join the pieces together.

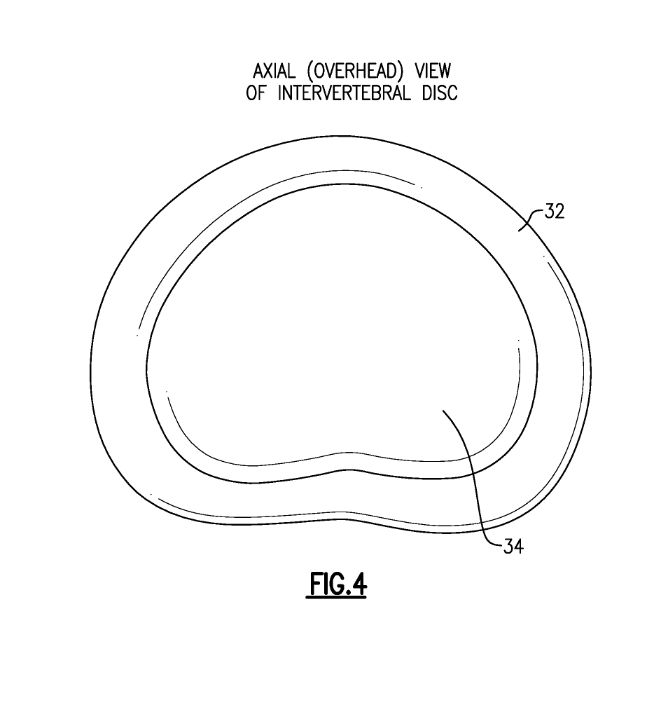

FIG. 4 is a schematic view of an intervertebral disk 30 that can be made from spacer fabric 10. The spacer fabric 10 is designed to act as an intervertebral disk and provide cushioning between adjacent vertebrae. The spacer fabric 10 may be homogenous in mechanical properties or it may have regions of differing mechanical properties. When the spacer fabric 10 is made of a shape memory material, the spacer fabric 10 is highly crush resistant. The spacer fabric intervertebral disk 30 can be placed directly adjacent to a vertebrae or it can be fixed to plates that engage the adjacent vertebrae.

Different filament materials, different densities, and different diameter filaments can be used to produce the intervertebral disk 30. Therefore, the knit material, the density, and the filament size may vary across the intervertebral disk 30. In one example, the intervertebral disk 30 has a very tough outer annulus fibrous 32 and a nucleus pulposus 34. The annulus fibrous 32 may be made of a bio-resorbable material. The removed annulus fibrous 32 may be ground up and impregnated into the disk to promote regrowth. The spacer fabric intervertebral disk 30 may be partially resorbable and partially non-resorbable.

In one example, the spacer fabric 10 is formed out of a shape memory material. In another example, the shape memory material comprises Nitinol.

In an embodiment according to any of the previous embodiments, a device for promoting osseointegration between two adjacent vertebrae includes a spacer fabric includes a top layer, a bottom layer, and intermediate filler fibers connecting the top layer and the bottom layer. The spacer fabric is capable of expanding to fill a gap between two adjacent vertebrae.

In another embodiment according to any of the previous embodiments, the spacer fabric is formed out of a shape memory material.

In another embodiment according to any of the previous embodiments, the shape memory material comprises Nitinol.

In another embodiment according to any of the previous embodiments, the spacer fabric is formed out of a polymer.

In another embodiment according to any of the previous embodiments, the polymer includes polyether ether ketone.

In another embodiment according to any of the previous embodiments, the spacer fabric is a knit or a weave.

In another embodiment according to any of the previous embodiments, the device is an intervertebral fusion device.

In another embodiment according to any of the previous embodiments, the spacer fabric is made of the spacer fabric and impregnated with a biologically active agent.

In another embodiment according to any of the previous embodiments, the biologically active agent includes hydroxyapatite or calcium phosphate.

In another embodiment according to any of the previous embodiments, the intervertebral fusion device is made of polyether ether ketone and coated with the spacer fabric.

In another embodiment according to any of the previous embodiments, the spacer fabric is a rolled cylinder that acts as a spinal cage.

In another embodiment according to any of the previous embodiments, the rolled cylinder of the spacer fabric is inserted into a rigid cage.

In another embodiment according to any of the previous embodiments, the device is an intervertebral fusion cage comprising polyether ether ketone that has a coating of the spacer fabric.

In another embodiment according to any of the previous embodiments, the device is an intervertebral disk made of the spacer fabric.

In another embodiment according to any of the previous embodiments, different regions of the implantable synthetic intervertebral disk are fabricated from different materials.

In another embodiment according to any of the previous embodiments, different regions of the implantable synthetic intervertebral disk are fabricated from filaments having a different diameter.

In another embodiment according to any of the previous embodiments, different densities of filaments of the implantable synthetic intervertebral disk are used in different regions of the implantable synthetic intervertebral disk.

In another embodiment according to any of the previous embodiments, at least some of the filaments are bioabsorbable.

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A device for promoting osseointegration between two adjacent vertebrae, the device comprising:
a spacer fabric including a top layer, a bottom layer, and intermediate filler fibers connecting the top layer and the bottom layer, wherein the spacer fabric is capable of expanding to fill a gap between two adjacent vertebrae.

2. The device according to claim 1 wherein the spacer fabric is formed out of a shape memory material.

3. The device according to claim 2 wherein the shape memory material comprises Nitinol.

4. The device according to claim 1 wherein the spacer fabric is formed out of a polymer.

5. The device according to claim 4 wherein the polymer comprises polyether ether ketone.

6. The device according to claim 1 wherein the spacer fabric is a knit or a weave.

7. The device according to claim 1 wherein the device is an intervertebral fusion device.

8. The device according to claim 7 wherein the spacer fabric is made of the spacer fabric and impregnated with a biologically active agent.

9. The device according to claim 8 wherein the biologically active agent includes hydroxyapatite or calcium phosphate.

10. The device according to claim 7 wherein the intervertebral fusion device is made of polyether ether ketone and coated with the spacer fabric.

11. The device according to claim 1 wherein the spacer fabric is a rolled cylinder that acts as a spinal cage.

12. The device according to claim 11 wherein the rolled cylinder of the spacer fabric is inserted into a rigid cage.

13. The device according to claim 1 wherein the device is an intervertebral fusion cage comprising polyether ether ketone that has a coating of the spacer fabric.

14. The device according to claim 1 wherein the device is an intervertebral disk made of the spacer fabric.

15. The device according to claim 14 wherein different regions of the implantable synthetic intervertebral disk are fabricated from different materials.

16. The device according to claim 14 wherein different regions of the implantable synthetic intervertebral disk are fabricated from filaments having a different diameter.

17. The device according to claim 14 wherein different densities of filaments of the implantable synthetic intervertebral disk are used in different regions of the implantable synthetic intervertebral disk.

18. The device according to claim 17 wherein at least some of the filaments are bioabsorbable.
